(19) United States
(12) Patent Application Publication
(10) Pub. No.: US 2013/0172689 A1
(43) Pub. Date: Jul. 4, 2013

(54) Emergency Medical Diagnosis and Communications Device

(71) Applicants: Vasilios D. Dossas, Chicago, IL (US); Clifford H. Kraft, Naperville, IL (US); Lady C. Dy, Chicago, IL (US)

(72) Inventors: Vasilios D. Dossas, Chicago, IL (US); Clifford H. Kraft, Naperville, IL (US); Lady C. Dy, Chicago, IL (US)

(21) Appl. No.: 13/557,667

(22) Filed: Jul. 25, 2012

Related U.S. Application Data
(63) Continuation of application No. 12/683,912, filed on Jan. 7, 2010, now abandoned.
Application No. 12/683,912 is a continuation of application No. 11/260,668, filed on Oct. 27, 2005, now abandoned.

(51) Int. Cl. A61B 5/00; A61B 5/01; A61B 5/0402; A61B 5/0205; A61B 5/145; A61B 5/15; A61B 5/151; A61B 5/157; A61B 5/053; A61B 5/11; A61B 5/04

(52) U.S. Cl. CPC A61B 5/6898; A61B 5/7264; A61B 5/1112; A61B 5/01; A61B 5/0402; A61B 5/02055; A61B 5/04012; A61B 5/443; A61B 5/150389; A61B 5/151; A61B 5/157; A61B 5/0537; A61B 5/0531; A61B 5/14542; A61B 5/749; A61B 5/741; A61B 5/14539; A61B 5/14532; A61B 5/14546
USPC 600/301; 600/300; 600/490; 600/500; 600/549; 600/509

(57) ABSTRACT

A portable emergency medical device capable of communicating with a remote location preferably as a cellular telephone that can measure one or more human vital parameters such as pulse rate, body temperature, skin moisture, blood pressure, ECG or blood chemistry and can receive symptoms from a user either by voice recognition or by keypad and can provide an expert medical diagnosis. The device can store a complete medical history for one or more users and can use an expert system to make the diagnosis. The device can make an emergency medical call either on command or automatically requesting help and optionally supplying medical information and/or GPS location information.

This application is a continuation of application Ser. No. 12/683,912 filed Jan. 7, 2010 which was a continuation of application Ser. No. 11/260,688 filed Oct. 27,2005. Application Ser. Nos. 12/683,912 and 11/260,688 are hereby incorporated by reference.

### BACKGROUND

1. Field of the Invention

The present invention relates generally to emergency medical diagnosis and more particularly to a handheld emergency medical diagnosis and communications device or agent.

2. Description of the Prior Art

Many travelers experience symptoms of serious illness when traveling and away from home. It is also known that symptoms for many serious medical events occur at night when medical consultation is difficult to obtain without calling 911 or reporting to an emergency room. In some cases the symptoms signal the onset of very dangerous medical conditions that require immediate help such as a myocardial infarction (heart attack). Other times, the symptoms relate to a relatively minor medical condition (such as an upset stomach or influenza).

Anzellini et al. in U.S. Pat. No. 6,339,720 teach an early warning apparatus for acute myocardial infarction. This apparatus is a portable electrocardiograph (ECG) that records and compares the ST segment of the patient's heart waveform with templates to decide if a heart attack is underway. U.S. Pat. No. 6,339,720 is hereby incorporated by reference.

Robinson et al. in U.S. Pat. No. 6,771,172 teach a portable patient monitor with an alarm while Ma et al. in U.S. Published Application number 2005/0203353 teach a multiple purpose portable apparatus for measurement analysis and diagnosis that evaluates test samples of a lateral flow or microplate assay.

It would be advantageous to have a handheld device that could perform critical medical tests such a pulse, ECG, emergency blood chemistry, blood pressure, body temperature and other tests to produce an immediate medical diagnosis and give an opinion as to what action should be taken. It would be further advantageous if the device contained a cellular telephone or other communications circuits (or was a cellular telephone) that could make a 911 call if requested or could make that call automatically if the patient did not respond to prompts and the diagnosis was serious. Such a device could contain a GPS receiver (such as those installed in many cellular telephones today) that could allow emergency medical personnel to locate the device.

### SUMMARY OF THE INVENTION

The present invention relates to a handheld medical diagnosis device that either is a cellular telephone or contains a cellular telephone or other communications circuits that can run a panel of medical tests measuring one or several vital parameters of the user, and using an expert system or other reasoning system, give an immediate emergency diagnosis to allow manual or automatic calling of emergency medical personnel if required. The device can be personalized and contain a complete medical history and record for people who might use it (such as members of a family). While a medical history helps make a more accurate diagnosis, the device can produce a diagnosis with or without the medical history. The device, in a preferred embodiment, can carry on a voice conversation with the patient while data concerning symptoms is entered. Symptoms, recent activity (eating, exercising, etc.), tests (pulse, blood, ECG, etc.), and medical history can be combined using either a local or remote reasoning system such as an expert system to produce a diagnosis and suggest a course of action and/or place an emergency medical call. In the event of an emergency medical call, the device can optionally upload all of its test data to a central hospital system and make the data available to responding emergency personnel. The device can automatically make and emergency call if the diagnosis indicates a severe medical condition and the user has not made the call after a predetermined period. The device can contain a GPS receiver to provide location information to responding emergency personnel.

### p DESCRIPTION OF THE FIGURES

FIGS. 1A and 1B show a front and side view of an embodiment of the present invention as a cellular telephone.

FIG. 2 shows the embodiment of FIGS. 1A-1B with the back open showing measurement components.

FIG. 3 shows some of the internal medical components of the embodiment of FIGS. 1A, 1B and 2.

FIG. 4 shows a detail of a finger measurement compartment.

FIG. 5 shows a detail of a blood chemistry lab chip.

FIG. 6 shows a block diagram of an embodiment of the present invention.

FIG. 7 shows a typical diagnostic procedure flow chart.

FIG. 8 shows a normal ECG pattern.

Several figures and illustrations have been presented to better aid in the understanding of the present invention. The scope of the present invention is not limited to what is shown in the figures.

### DESCRIPTION OF THE INVENTION

The present invention relates to an emergency medical device or agent coupled to a portable communications device like a cellular telephone. In a preferred embodiment, the medical device and a cellular telephone share a single housing; however, it is within the scope of the present invention for the communications device and the medical agent to occupy separate housings that are coupled electrically and/or wirelessly and/or mechanically.

The present invention is particularly useful for diagnosing medical conditions that occur on vacation or business trips, at home on weekends or at night, or at times when it is not convenient to call a family physician. In particular, the medical agent can measure and sample several human physiological parameters such as body temperature, skin moisture, pulse rate, blood pressure and can take an electrocardiogram (ECG), measure blood pressure and perform blood chemistry as well as gather symptoms by either voice recognition or from a keypad, access the person's medical history, make a diagnosis, and suggest either going to an emergency room or waiting. The communications device can call emergency medical personnel if necessary. In one mode of operation, the present invention can suggest an alternative of either going to an emergency room or calling 911. If the patient does not do either, the device can, after a predetermined period of time, automatically call 911. The communications device can contain a GPS receiver that allows emergency personnel to immediately find it.

In a preferred embodiment, the emergency medical device or agent is part of a GPS-equipped cellular telephone. On power-up, the telephone acts as any other cellular telephone allowing the user to make and receive telephone calls, browse the internet, etc. However, upon pushing a special key, or entering a certain sequence on the keypad or voicing a command, the device can enter a medical mode. In this mode, the person's physiological parameters can be taken, and symptoms can be acquired either by voice in a question and answer format, freeform, or via a keypad and display. A user's complete medical history can be stored in the device and be consulted. An expert diagnosis system, or other reasoning system, can use the medical history and the physiological parameters to ask for specific symptoms and to walk through questions and answers regarding symptoms. The system can then make a diagnosis and recommend action.

An example of an expert symptom gathering exchange might be: “What is your major symptom?” “A pain in my stomach.” “Is the pain higher or lower or at the same level as your belly button?” “Same level.” “Is the pain on the right, center or left?” “Right”. “Is it a sharp pain?” “Yes”. “How long has it been bothering you?” “All day.” “Is it getting worse?” “Yes.” “How long ago did you eat?” “2 hours ago, but I couldn't eat much.” “Do you feel nauseated?” “Slightly.” “Have you vomited?” “No.” “Please place your finger in the analysis chamber so I can read your pulse and temperature.” “Okay, please put the wrist cuff on so I can read your blood pressure.” “Looking at your medical history and noting that you have a fever of 102 degrees, damp skin and somewhat reduced blood pressure, there is a possibly this is your appendix.” “While it might be something you ate, I highly suggest you either report to an emergency room or have me call 911 because of the danger of an infected appendix.” “Which do you prefer.” “I will catch a cab to the emergency room.” “Okay; however, when you arrive, enter code 63 or I will automatically call 911 after ½ hour.” This exchange is an example of a possible session the preferred invention might have with a user. Any type of exchange is within the scope of the present invention.

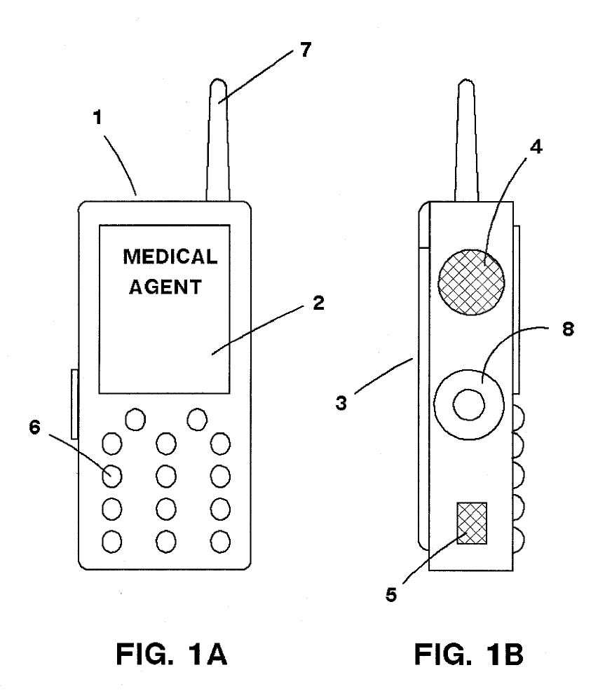

The present invention can take the form of a cellular telephone or pager in a preferred embodiment. Turning to FIGS. 1A and 1B, a cellular telephone/medical agent can be seen. A housing 1 contains a display 2, a keypad 6, an antenna 7, a speaker 4 and microphone 5. The device normally operates as a cellular telephone, pager, browser, walky-talky or any other communications device. However, the embodiment shown in FIG. 1B contains a back panel 3 that can open and a chamber 8 for measuring various physiological parameters. The chamber 8 can optionally have a removable, sliding or hinged cover for protection.

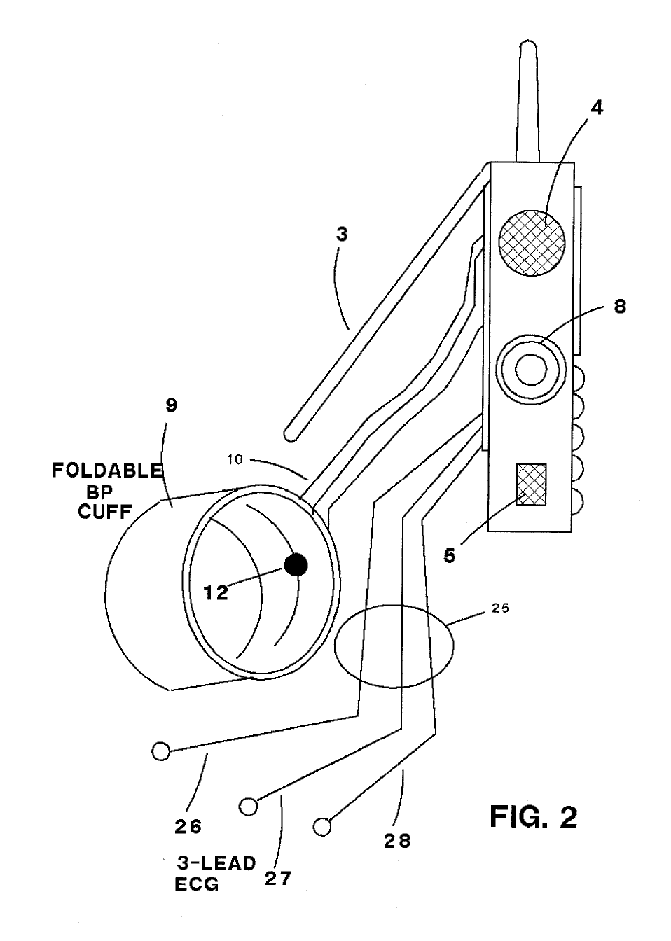

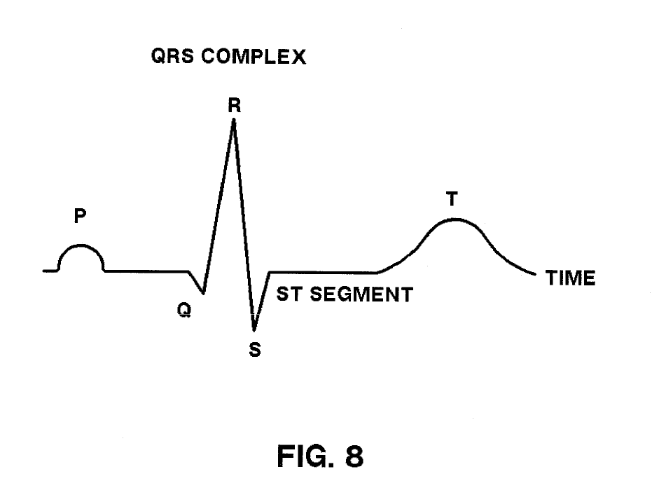

The back panel 3 in this embodiment can open to allow access to several test devices as can be seen in FIG. 2. A foldable wrist cuff 9 driven by a cable and air tube 10 and several ECG leads 25 can fold out of a compartment that is normally closed by the panel 3. The wrist cuff 9 can be used to estimate blood pressure, while the ECG leads can be used to perform a 3-lead ECG with one lead 26 being placed on the left chest/armpit, a second lead 27 being placed on the right chest/armpit, and a return lead 28 being placed in the center on the lower abdomen. While FIG. 2 shows a wrist cuff, any other device or method for measuring or estimating blood pressure is within the scope of the present invention including a finger blood pressure measuring system contained in the test chamber 8. Also while FIG. 2 shows three ECG leads, any number of ECG leads is within the scope of the present invention. A normal ECG signal that might be expected with a 3-lead ECG is shown in FIG. 8.

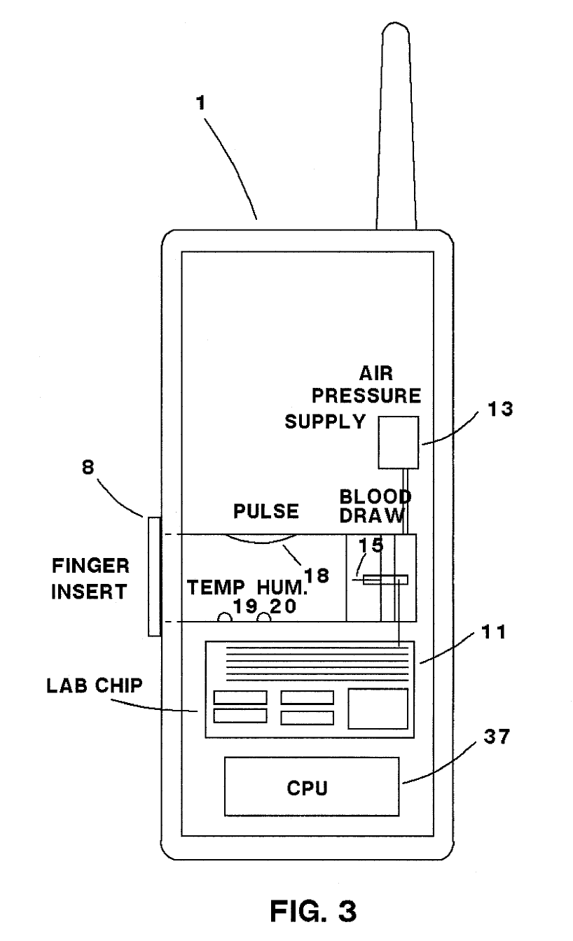

Turning to FIG. 3, some of the internal parts of the medical agent can be seen. The chamber 8 is designed for the insertion of a finger (this could be modified to receive a toe or other member for patients who have no fingers). The chamber 8 can contain a body temperature sensor 19, humidity or skin dampness sensor (normally skin resistance) 20 and pulse rate sensor 18. In addition, a possibly hollow, micro-lance 15 can be used to obtain a drop of capillary blood by means of a finger stick. Below the chamber 8 in FIG. 3, a laboratory on a chip (lab chip) 11 can be seen. This type of chip can perform a very detailed blood chemistry analysis. A CPU 37 is also seen in FIG. 3. This processor can be separate or part of the telephone MPU processor. This processor can control all medical procedures and analyze results to produce a diagnosis.

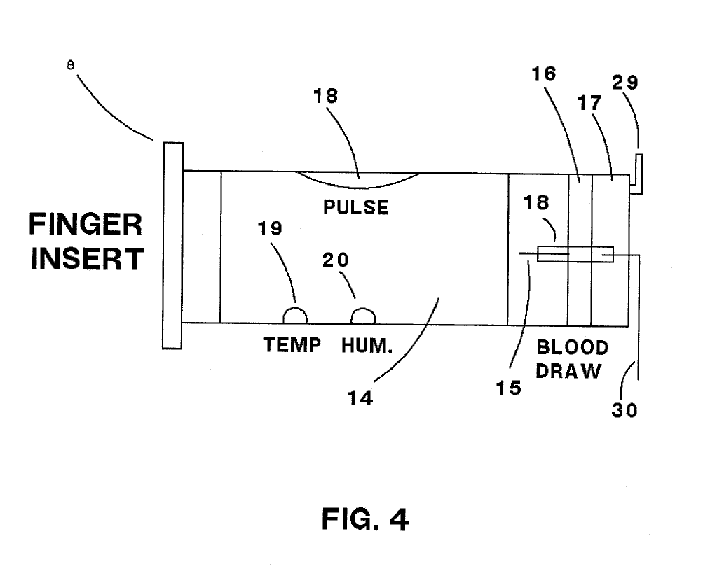

A detail view of the finger-test chamber 8 is shown in FIG. 4. The chamber contains several sensors. A pulse rate sensor 18 can be a small accelerometer or pressure sensor that feeds back a pulse pressure waveform to an interface. A timer/counter (not shown) can convert that to a standard pulse rate of N beats per second. A skin temperature sensor 19 can be used to measure the temperature of the finger. This sensor can come to a fast equilibrium and provide a value that can be used to accurately estimate core body temperature. Because extremities such as the finger may not receive as much total blood quantity as an interior area such as under the tongue or the rectum, the read finger temperature can be lower then the real core body temperature. An interface or processor can offset the finger temperature by a fixed amount to estimate core body temperature. Since circulation in females may be less than males, the offset can optionally be adjusted according to the gender of the user. Gender will be known if the user has supplied their medical history. Also, the user can be ask gender during the symptom gathering process. A skin moisture sensor 20 (marked HUM in FIG. 4) can provide information on skin dampness. This sensor can generally be a skin resistance sensor. It is known in the art that wet skin can have a resistance as low as 5 K ohms or less and that dry skin can have a resistance or up to several hundred K ohms. Skin moisture can be based on a relative scale related to electrical skin resistance.

FIG. 4 also shows a blood draw needle or lance 15. In the preferred embodiment, this device is a hollow needle of capillary size that can slide in and out in a sheath 18. The needle can be caused to stick the finger under control of air pressure supplied through a pressure source tube 29 coupled through a wash chamber 16. The first step in a blood draw can be to eject a few micro-liters of a sterilization wash fluid through the needle from the inside. Next the needle can be totally evacuated by reverse pressure. Finally the sterile, empty needle can be forced forward into the finger by air pressure. Capillary action or reverse pressure can be used to draw a small amount of blood. This blood sample can be conveyed through a micro-conduit 30 to a capillary in a blood chemistry chip. After the blood draw, the needle can be re-washed. Optionally, before the actual stick, alcohol or other suitable disinfectant, possibly the wash fluid, can be sprayed on the tip of the finger. While the preferred embodiment contains a pressure controlled finger-stick needle, any method or device for taking a blood sample is within the scope of the present invention. A replaceable, throw-away lance is also within the scope of the present invention.

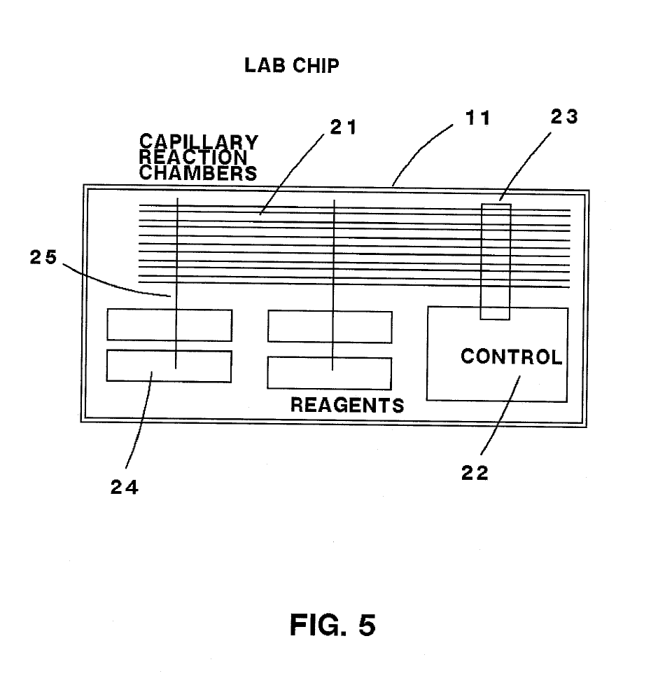

FIG. 5 shows an embodiment of a blood chemistry chip 11. Micro laboratory chips are known in the art. The lab chip shown in FIG. 5 contains a large number of capillary reaction chambers 21 where portions of a blood sample can be routed for analysis. Each different test may require a separate capillary. Modern lab chips can contain thousands of such capillaries. Various reagents can be stored in reagent chambers 24 and steered into particular capillaries as needed by micro-fluidic methods known in the art. A control unit or processor 22 can be used to control micro-valves and pumps 23 to route blood and reagents. Various readout methods can be used including light absorption and fluorescence. A light source and/or detector can be mounted external to the chip or integrated onto the chip. Any detection system or method is within the scope of the present invention. The reaction chip can be programmed to perform any number of standard blood chemistry tests. Tests that provide immediate emergency diagnostic information are preferred as will be explained.

Returning to FIG. 2, a blood pressure cuff 9 can be seen. This cuff can generally fold up and be stored in the back of the device. Unfolded, this cuff can be large enough to fit a human wrist. The cuff can be inflated and controlled by an air pressure tube 10 that causes it to contract and release. Pressure can be supplied by a miniature pump that can be mounted in the housing. A sensor 12, which can be acoustic or pressure, can be mounted in the cuff. This sensor, operating in the normal way, can detect wrist pulse sounds to establish both a systolic and diastolic pressure. The manner of operation is known in the art. The cuff is tightened to a predetermined amount and the sensor 12 begins listening for a pulse sound. The cuff is released in small steps while a pressure sensor in the housing reads pressure. The systolic blood pressure point is established when the pulse is first heard. The cuff is further released until all pulse sound stops. This is the diastolic pressure point. The pressure in the cuff is then totally released for removal. It is known in the art that blood pressure taken at the wrist may read differently than pressure taken on the arm. For this reason, it is desirable to adjust the reading to more accurately reflect the actual blood pressure.

FIG. 2 also shows a 3-lead ECG arrangement 25. It is known in the art to place a leads near each armpit 26, 27 with a return lead on the lower abdomen 28. It is also known in the art for a processor to analyze this 3-lead ECG data to establish a P wave, QRS complex, ST segment and T wave if possible. While numerous different analyses of ECG waveforms are possible and known in the art, it is particularly useful in a possible emergency situation to establish the positive or negative displacement of the ST segment. FIG. 8 shows a normal ECG waveform. If the ST segment is displaced from the 0 voltage point plus or minus by more than 100 microvolts, there is a possibility of a myocardial infarction. A displacement of more than 200 microvolts shows the possibility of a particularly dangerous situation. The present invention can instruct the user to immediately report to an emergency room in the case of any offset more than 100 microvolts of an established ST segment. Optionally, the device of the present invention can manually or automatically place a 911 call in any extreme case or by request of the user. The method for examining the offset of the ST segment from such a 3-lead ECG is explained in U.S. Pat. No. 6,339,720. Of course, before such a diagnosis can be made, the ECG signal must establish a solid ST segment detection. If the signal cannot be read (for example, the QRS complex cannot be detected), the user can be instructed to apply extra conductive cream to the leads. This cream could be optionally supplied in the device case.

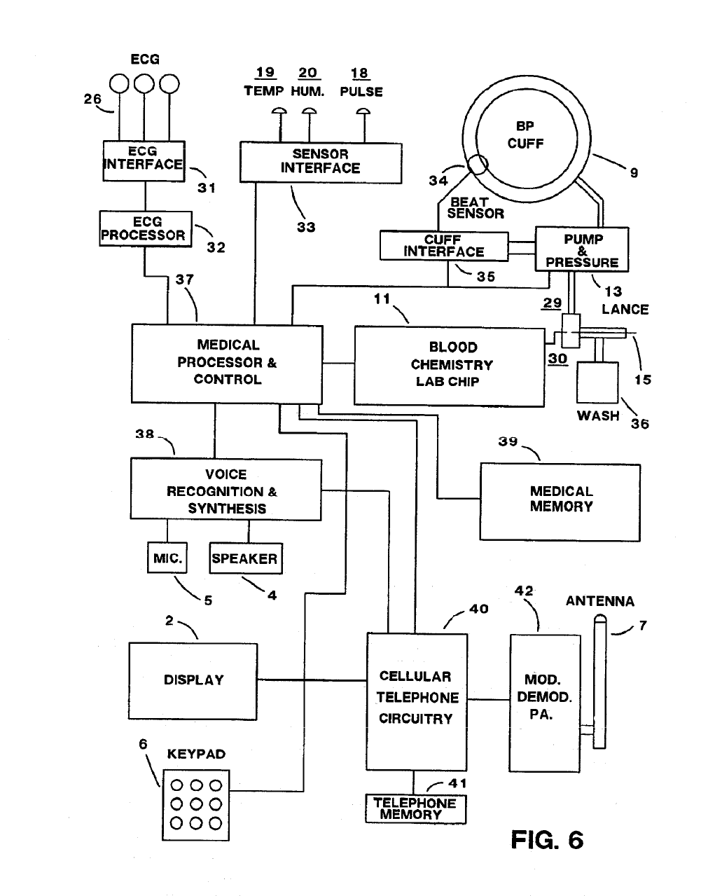

Turning to FIG. 6, a block diagram of an embodiment of the present invention is shown. A medical control processor 37 controls all medical testing. This processor can be the same processor as the telephone MPU, or more likely, a separate processor dedicated to medical tasks. In any case, this processor is normally connected to the telephone or communications circuitry 40 so that it can initiate telephone calls or other communication and can log onto a particular web site or other communications program or server if diagnostic processing is performed offline. While the preferred method is to perform all diagnostic processing onboard the device, it is within the scope of the present invention to perform all or some of the processing offline or to offload information for an offline human or automatic diagnosis. If diagnostic processing is performed onboard, the medical processor 37 can run a decision or reasoning program such as an expert system, rule inference engine, or any other type of artificial intelligence program. The processor 37 can be coupled to a medical memory 39 that can store programs, algorithms and medical histories for one or more users.

The medical processor 37 can interface with a blood chemistry chip 11, a medical memory 39, optional voice recognition and synthesis unit 38, and communications circuitry such as a cellular telephone transceiver 40. The voice unit 38 can be connected to the telephone speaker/earphone 4 and microphone 5 which are also accessible by the communications circuitry 40. In addition, the medical processor 37 can drive the pressure pump 13, the blood pressure cuff 9 with beat sensor 34 and cuff interface 35 as well as the temperature/moisture/pulse sensor interface 33. The medical memory 39 can be used to store medical histories of the user and others such as family members who might use the device. Software in the medical processor 37 can allow loading of medical histories into the medical memory 39. In an optional mode of operation, the present invention can communicate via the communications circuitry 40 with remote medical facility either directly or via the internet. Medical history could be optionally stored at this remote facility or not used.

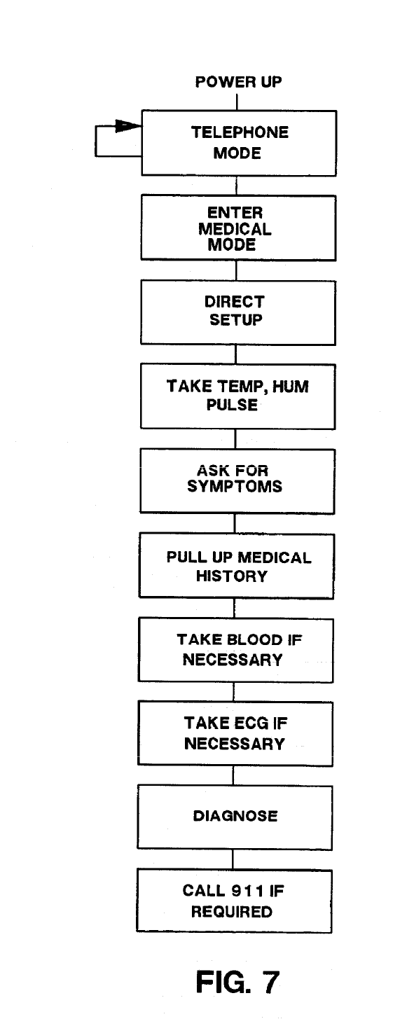

Typical operation of the present invention can be traced with a flow chart. Turning to FIG. 7, the device powers-up in telephone (or normal communication) mode. In this mode, normal telephone calls, browsing, email, short messaging, etc. can be performed. Upon entry of a key or voice command, or code, the device can switch to medical mode. Here the patient can be directed to open the device and set up for various medical tests. Tests can be run (temperature, pulse, etc.) and symptoms can be entered. Optionally, symptoms can be entered before any testing is done. The user's medical history can be consulted (if stored), and a decision can be made whether tests like ECG, blood pressure and/or blood chemistry are needed. If ECG is indicated, the patient can be instructed to attach the ECG leads to his or her chest. If blood pressure is indicated, the patient can place the cuff around his or her wrist, and if blood chemistry is needed, the patient can be so-advised, and a finger stick can be performed. After all symptoms are entered (possibly iteratively) and all tests are performed, a diagnosis can be reported (and/or printed if a printer is attached). The diagnosis can be stored for future reference, uploaded to a medical site if desired (or required), and can be shown on a display. In any serious condition, the patient can be told to either report to a medical facility for help or call 911. The device can offer to call 911, and can make the call automatically if there is no response or after a predetermined time. A GPS receiver in the device can aid emergency personnel to locate the device. This can be a stand-alone GPS or an assisted GPS known in the art.

As an emergency mode, the device of the present invention can enter medical mode whenever a finger is inserted into the test canal. This feature allows diagnosis and emergency calling in cases where the person is in too much pain to do more or cannot talk. The present invention can also call 911 immediately when a particular duress key is pushed or a particular voice command is spoken.

When the device of the present invention performs blood chemistry tests, various different tests are possible. Of primary interest are those that can be done in a portable unit without human intervention and that may help formulate a correct diagnosis in an emergency situation. Of particular interest are those blood tests that might point to a myocardial infarction (heart attack). Of secondary interest are tests such as blood sugar and electrolytes. Possible blood chemistry tests may include blood oxygen, blood oxygen saturation, blood carbon dioxide, blood pH, total CK, CK-MB, AST, myoglobin, BUN, serium ketones, blood electrolytes and blood glucose as well as blood electrolytes. For example, total CK is known to increase within 3-6 hours after the onset of an infarction, CK-MB 4-8 hours AST 6-8 hours and myoglobin 2-3 hours. Another possibility is CK-MB-2/MB-1 which is known to increase after about 2 hours. CK-MB is one of the more reliable tests known to have a specificity of greater than 93% and a sensitivity of greater than 94%. Blood gas chemistry can indicate whether there is a respiratory or other problem with O2/CO2 exchange. Optionally, the present invention can perform a complete blood count detecting various blood cell problems including detecting a decrease in hemoglobin reflecting possible hemorrhage; rise in white cell count for infection and rise in BUN indicating dehydration. Optional chemistry could test for drug levels of commonly taken drugs such as for overdose.

The present invention generally takes capillary blood for chemical analysis. For some tests, a correction may be needed. For example, it is known the pO2 (percentage blood oxygen) is usually read lower in capillary blood compared to arterial blood (45-60 compared to 80-100). Oxygen saturation is usually around 70% compared to 95% for arterial blood.

In the case of diabetes mellitus, a medical history check may show that the user has this disease. In any case a blood glucose reading greater than 200 mg/dL indicates a dangerous condition of hyperglycemia that requires immediate attention. On the other hand, blood glucose of less than 10 mg/dL indicates severe hypoglycemia which also requires immediate attention. In the hyper case, blood ketones will also normally be elevated.

Several scenarios are presented as examples of diagnoses of possible problems that might occur at home, at night, on the road, to an owner of the device or to a family member. These are examples only; many other possible diagnoses can be made, and the diagnoses of these examples might be slightly different. Any diagnosis, base on any symptoms and/or human vital parameters is within the scope of the present invention.

ANY SHARP OR DEBILITATING PAIN WITH NO ASSOCIATED INJURY SHOULD ALWAYS RESULT IN A CALL FOR MEDICAL HELP.
Myocardial Infarction: squeezing or crushing retrosternal pain possibly radiating to neck, jaw or left arm (sometimes right arm). Similar discomfort of lesser magnitude in proceeding hours or even days. Raised or lowered ST segment and/or peaked T wave in ECG., possibly increased total CK, CK-MB or myoglobin levels. Medical history may indicate previous cardiac problems or infarctions. Immediate emergency help is imperative.
Cardiac Arrhythmias: Pulse rates of greater than 120-140 beats/min. or less than 60 beats/min. with normal ECG ST segment and T wave may indicate tachycardia or bradyacardia respectively (however, check for myocardial infarction should always be made). Lowered cardiac output may cause cold, clammy skin. Medical history may show history of heart irregularities. If patient has a pace-maker, medical help should be immediately sought. Age may be a factor. A patent with a history of mild tachycardia can rest and wait; however, if condition persists longer than 1 hour, or worsens, or if severe symptoms are present, medical help should be sought. If ECG abnormal other than rate, medical help should be sought.
Dizziness, Shock: Low blood pressure, high skin moisture level, fast pulse rate (shock), rapid shallow breathing, ECG normal, no pain. No other abnormalities noted. For slight dizziness, the patient can rest flat on back with legs raised. If condition persists more than a few minutes, or there is a fever, medical help should be sought. Fever and/or chills, along with other shock symptoms may indicate septic shock; immediate medical attention is mandatory.
Appendicitis: Sharp pain in lower abdomen, center to right side. Possibly nausea, vomiting, and/or diarrhea. Pain shifts to lower right quadrant. Elevated temperature of 1 or 2 degrees. Seek immediate medical help.
Perforation of Peptic Ulcer: Sudden (occurring out of nowhere) severe prostrating pain in abdomen. Possible medical history of duodenal ulcer (or sometimes gastric ulcer). Pain may start in the epigastrium but rapidly spreads over entire abdomen. Patient very (critically) ill. Immediate medical help is imperative.
Food or other Poisoning: Nausea and vomiting, stomach pain, abdominal discomfort, maybe diarrhea, subnormal or raised body temperature without any other symptoms or history (such as flu), fast or slow pulse rate (usually fast), possible chest pain, shortness of breath and even confusion and seizures. Ask patient if any medications recently taken. Ask if meal recently eaten. Self-induced emesis (vomiting) can be effective for poisonings (by mechanical stimulation of the oropharynx). Medical help should be sought if condition remains more than 1 hour or after vomiting or if severe. A decrease in pCO2 may accompany some poisonings. Botulism causes sudden appearance of symptoms 8-36 hours after eating contaminated food such as nausea and constipation along with dry mouth. Within 24 hours these symptoms are followed by muscle weakness that starts in the eyes causing blurry vision and the progresses down the body. Botulism requires immediate medical intervention. Other food poisonings include staphylococci, E-coli, salmonella and campylobacter which cause mild to severe symptoms. Mild cases of these types of food poisoning usually clear up within from 1-3 days. Severe symptoms require medical help. Optional chemistry can detect overdose levels of commonly taken drugs such as acetaminophen, insulin, aspirin, digoxin and others.
Diabetic History—Hyper/Hypoglycemia: Headache, irritability, dizziness, weakness, fainting, impaired cognition. Blood glucose elevated (>150-200 mg/dL), and person takes insulin, injection should be given. Re-test within 30 minutes. Blood glucose depressed may indicate insulin overdose in diabetic—take sugar pill or sugar in orange juice. (Symptoms of both hyper and hypoglycemia may be similar). Low blood glucose in non-diabetic may be caused by liver failure (check history for alcohol abuse), or toxic dose of aspirin or acetaminophen (ask if any drugs of any type recently taken).
Pneumonia and Legionnaires' Disease: Rapid onset of symptoms of chest pain that becomes worse upon inhalation, cough that may produce rust-colored or bloody sputum, shortness of breath at rest, high fever even delirium or confusion. Any type of pneumonia requires immediate medical intervention.
Mononucleosis: High fever and sweating, extremely sore throat, possibly causing difficulty swallowing, swollen tonsils, enlarged tender lymph nodes in neck armpits and groin, possibly tender abdomen. Drink lots of cool fluid and take over-the-counter analgesic such as acetaminophen. If fever greater than 103 degrees, medical help should be sought.
Hemorrhage from perforated bowel or bleeding ulcers: Dizziness, nausea, passing out, low blood pressure, shock symptoms, low blood hemoglobin. Needs immediate medical care.
Stroke: blurred vision, disorientation, inability to speak, partial paralysis, possibly low blood pressure. Needs immediate medical care.

While particular examples of diagnoses have been given to illustrate possibilities for the present invention, numerous other results and test combinations may be indicated by medical history and/or symptom combinations, and numerous other diagnoses may be made by the system of the present invention.

The reasoning system must generally consider some of the following factors in forming a diagnosis.

A diagnosis of myocardial infarction (heart attach) rests on the history of prolonged chest discomfort, electrocardiographic changes consistent with ischemia or necrosis and elevated cardiac enzymes. The emergency medical device of the present invention can ask for symptoms such as type, location and intensity and duration of pain, along with electrocardiographic changes and elevated cardiac enzyme levels, a probable or definitive diagnosis of myocardial infarction can be rendered thereby prompting the prompting the patient to seek emergent medical care. In addition the medical device can promptly instruct patients to initiate simple measures to help relieve ischemic pain, restoring early blood flow to the heart hereby, reducing the overall severity of the heart attack.

Another potential application of the present invention is in the prompt management of acute heart failure that if not recognized early is associated with high morbidity and mortality. Clinical signs and symptoms such as fatigue, exercise intolerance, shortness of breath and leg swelling, in addition to elevated serum levels of BUN and creatinine, low sodium levels and elevated levels of liver enzymes are points that can be elicited by the medical device and based on the information provided, a recommendation to seek emergent care is made.

In case of pericaditis, where there is inflammation of the pericardium that can be associated with a wide variety of etiological factors. The chest pain can be easily mistaken for myocardial ischemia, however the pain intensifies with respiration. It is imperative to recognize this condition as this can lead to pericardial effusion (accumulation of fluid in the pericardial sac). Rapid accumulation of fluid can cause cardiac tamponade, eventually death so that immediate pericardiocenteses is required. Equally important to the detection of symptoms of dysrhythmias such as irregular heart beat/cardiac rhythm as previously discussed.

Another vitally important application of the present invention is in the management of diabetes. The device can enable monitoring of blood glucose levels and determine the pathological or critical blood glucose values that may lead to diabetic ketoacidos and or coma. In these instances, prompt medical care will be requested.

In cases of abdominal pain, recognizing the severity, location and duration of the pain are all important factors in determining the acute nature of medical condition. The device can ask for a careful history such as drug and medial history, associated symptoms of vomiting, protracted retching, weight loss, anorexia and possible bleeding. After physical findings such as rate of breathing, skin temperature, skin dampness, heart rate, oxygen blood saturation will be measured by this medical device. After synthesis of all these information, the medical device can warn patients of an impending infection, such as appendicitis, pancreatitis, acute abdominal hemorrhage or perforated bowel.

Continuous passage of loose or watery stools may herald the onset of food poisoning as in the ingestion of contaminated foods that can eventuate in dehydration and serum electrolyte imbalances. These situations require prompt attention.

Another possible application of the device is the detection of acute bronchospasm heralded by progressively worsening dyspnea (shortness of breath), cough, tachypnea, chest tightness and continuous wheezing for prolonged hours. The medical device can contain a sensor that measures the breathing rate, skin moisture suggesting diaphoresis and sensor attached to the finger tip measuring blood oxygen and carbon dioxide levels and blood pH levels. Critical values will prompt the medical device to urge you to seek emergency care.

Several descriptions and illustrations have been presented to better aid in understanding the present invention. One skilled in the art will recognize that many changes and variations are possible. Each of these changes and variations is within the scope of the present invention.

We claim:

1. A personal medical diagnosis apparatus comprising:
a cellular telephone module including a processor and memory;
a medical expert system disposed in said memory and executable on said processor;
a medical probe that measures at least one human medical parameter, said probe being in data communication with said medical expert system; and
an input device, including a keyboard and circuitry through which the medical expert system receives symptom data;
the expert system receiving imputed symptom data and at least one medical parameter and making a medical diagnosis.

2. The personal medical diagnosis apparatus of claim 1 further comprising a GPS receiver in communication with said processor, wherein said processor is adopted to report a geographic location of said medical diagnosis apparatus to a remote station.

3. The personal medical diagnosis apparatus of claim 1 further comprising a housing wherein said medical probe is at least one of:
a blood pressure sensor in or deployable from said housing;
a pulse rate sensor in or deployable from said housing;
a body temperature sensor in or deployable from said housing, or;
an ECG interface in or deployable from said housing.

4. The personal medical diagnosis apparatus of claim 2 further comprising a housing wherein said medical probe is at least one of:
a blood pressure sensor in or deployable from said housing;
a pulse rate sensor in or deployable from said housing;
a body temperature sensor in or deployable from said housing, or;
an ECG interface in or deployable from said housing.
